(19) United States
(12) Patent Application Publication
(10) Pub. No.: US 2022/0170937 A1
(43) Pub. Date: Jun. 2, 2022

(54) SERIAL BIOMARKER MEASUREMENT USING A COMPARATOR APPROACH

(71) Applicant: Rahul Puri, Marvin (US)

(72) Inventor: Rahul Puri, Marvin (US)

(21) Appl. No.: 17/441,207

(22) PCT Filed: Mar. 23, 2020
(86) PCT No.: PCT/US20/24188

§ 371 Date: Sep. 20, 2021

Related U.S. Application Data
(60) Provisional application No. 62/821,712, filed on Mar. 21, 2019.

(51) Int. Cl. G01N 33/574; C12Q 1/6886; G01N 30/72

(52) U.S. Cl. CPC G01N 33/57415; C12Q 1/6886; G01N 2030/027; C12Q 2600/158; G01N 30/7233

(57) ABSTRACT

Provided are methods for serial monitoring of a biomarker using a comparator approach. In some embodiments, the method include providing a first timepoint measurement with respect to a biomarker present in a first biological sample isolated from a subject; obtaining subsequent timepoint measurements for the same biomarker in subsequent biological sample isolated from the subject; retesting the immediately previous biological sample along with the subsequent timepoint biological samples to obtain a comparative change in the biomarker between time points; calculating a percent change and/or other metric in the biomarker between consecutive timepoint biological samples; and calculating the biomarker concentration at each time point with respect to the first time point measurement using the percent change and/or other metric of biomarker values between consecutive time point biological samples.

### CROSS REFERENCE TO RELATED APPLICATION

This application is based on and claims priority to U.S. of America Provisional Patent Application Ser. No. 62/821,712, filed Mar. 21, 2019, the disclosure of which is incorporated by reference herein in its entirety.

### TECHNICAL FIELD

The presently disclosed subject matter is directed generally to measurement of the concentration of a biological marker (biomarker) or an analyte (chemical species) in blood circulation with the intention of determining the onset, progress or remission of disease of the organ in which the biomarker or analyte originates. The presently disclosed subject matter thus provides in some embodiments methods of accurately determining the changes in the biomarker or analyte measurement and the methods are applicable to measurement techniques currently available to measure concentrations of analytes or biomarkers as well as any new technologies that could be developed in the future to measure the same. These measurement techniques include, but are not limited to, immunoassays (ELISA, RIA), real time or quantitative polymerase chain reaction (RT-PCR) and mass spectrometry.

### BACKGROUND

Blood has long been thought of as a window to a person's health since it picks up specific biomarkers and analytes from the various organs, tissues or cell type as it circulates through the body. This has lead to the development of simple blood tests that detect the presence or the change of biomarkers and analytes for routine diagnostic screening. Most blood tests compare the concentrations of analytes and biomarkers in an individual to a normal range established from healthy volunteers (called the Reference Interval) to make a clinical decision. However, often times it is the change in these levels from the individuals own “healthy” levels that has higher sensitivity in the detection of disease onset. Whether an individual should be evaluated against his or her own “healthy” level or a population based Reference Interval established from healthy volunteers is a function of the “index of Individuality” which is a ratio of the intra-individual biological variation to the between individual biological variation of the biomarker or analyte. Despite serial monitoring against an individual's healthy baseline is a better approach with high sensitivity for most analytes and biomarkers that have strong individuality (Carmen Ricós et al. (2009) Application of biological variation a review, Biochemia Medica, 19(3):2509), most laboratories continue to rely on the population based Reference Interval.

One of the challenges to the use of serial monitoring is the fact that analytical variation is a part of each measurement and is additive to the biological variation. The analytical variations are driven by various factors that are inherent with the method and can be quite significant. This results in inflating the change threshold for clinical decision-making, resulting in reduced sensitivity. For example, ELISAs and RT-PCR's depend on enzymatic reactions, which are known to be sensitive to pH, temperature and other chemicals that can interfere with the measurement technique. Even slight changes in reagents manufactured in different batches and/or tested under slightly different conditions can cause significant analytical variations.

Thus, there is a need in the art to provide methods to reduce the contribution of analytical variation in serial measurements and increasing the effectiveness of diagnostic screening tests. The present invention provides for methods that fulfill this need.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides methods for serial monitoring of biomarkers using a comparator approach. In some embodiments, the methods comprise providing a first timepoint measurement with respect to a biomarker present in a first biological sample isolated from a subject; obtaining subsequent (2^{nd}, 3^{rd}, 4^{th} etc.) timepoint measurements for the same biomarker in subsequent (2^{nd}, 3^{rd}, 4^{th} etc.) biological sample isolated from the subject; retesting the immediately previous biological sample with respect to the biomarker along with the subsequent timepoint biological samples to obtain a comparative change in the biomarker between time points (i.e., 1^{st} sample retested with 2^{nd} sample, 2^{nd} sample retested with 3^{rd} sample, 3^{rd} sample retested with 4^{th} sample, etc.); calculating a percent change and/or other metric in the biomarker between consecutive timepoint biological samples (i.e., 1^{st} sample retested with 2^{nd} sample, 2^{nd} sample retested with 3^{rd} sample, 3^{rd} sample retested with 4^{th} sample, etc.); and calculating the biomarker concentration at each time point with respect to the first time point measurement using the percent change and/or other metric of biomarker values between consecutive time point biological samples. This approach of using the previous timepoint sample as a comparator minimizes the effect of analytical measurement variation due to different batches of reagents or differences in testing conditions between the samples.

In some embodiments, the methods further comprise outputting the percent change and/or other metric of biomarker values to a visible medium.

In some embodiments, the biomarker is an expression level of a gene product, optionally, a protein, peptide, nucleic acid, or a fragment thereof or is an amino acid or nucleic acid sequence thereof.

In some embodiments, the methods employ an ELISA assay or other assay.

Thus, it is an object of the presently disclosed subject matter to provide methods for serial monitoring of biomarkers that accurately measure serial changes in levels of biomarkers with strong individuality in individuals using a comparator approach.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE FIGURES

FIG. 1 illustrates the approach/process of testing, measuring, calculating and reporting of the biomarker results at various time points for an individual using their previous timepoint sample as a comparator on the same ELISA plate.

FIGS. 2A and 2B are graphs that compare the biomarker trend lines in a couple of patients using the comparator method against the conventional methods. The first patient (FIG. 2A) was being treated for breast cancer and the second patient (FIG. 2B) was being monitored for breast cancer recurrence.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

All references listed below, as well as all references cited in the instant disclosure, including but not limited to all patents, patent applications and publications thereof, scientific journal articles, and database entries are incorporated herein by reference in their entireties to the extent that they supplement, explain, provide a background for, or teach methodology, techniques, and/or compositions employed herein.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed lo subject matter.

The presently disclosed subject matter relates to a process to accurately measure the trend (increasing or decreasing) with time of biomarkers in patients. Clinicians often use the trends to guide treatment decisions and the current laboratory methods that rely on external materials as quality control are often not suitable for accurately determining the trends depending on the variability in the measurement system compared to the biological variability of the biomarker in the individual. The presently disclosed subject matter employs a patient's own sample from a previous timepoint as a comparator control and has demonstrated significantly higher accuracy in measured biomarker trendlines.

### II. Embodiments

The presently disclosed subject matter relates in some embodiments to methods for serial monitoring of biomarkers using a comparator approach. In some embodiments, the methods comprise (a) providing a first timepoint measurement with respect to a biomarker present in a first biological sample isolated from a subject; (b) obtaining one or more subsequent (e.g., 2^{nd}, 3^{rd}, 4^{th}, etc.) timepoint measurements for the same biomarker in subsequent (e.g., 2^{nd}, 3^{rd}, 4^{th}, etc.) biological sample isolated from the subject; (c) retesting the immediately previous biological sample with respect to the biomarker along with the subsequent timepoint biological samples to obtain a comparative change in the biomarker between time points (e.g., 1^{st} sample retested with 2^{nd} sample; 2^{nd} sample retested with 3^{rd} sample; 3^{rd} sample retested with 4^{th} sample; etc.); (d) calculating a percent change and/or other metric in the biomarker between consecutive timepoint biological samples (1^{st} sample retested with 2^{nd} sample; 2^{nd} sample retested with 3^{rd} sample; 3^{rd} sample retested with 4^{th} sample; etc.); and (e) calculating the biomarker concentration at each time point with respect to the first time point measurement using the percent change and/or other metric of biomarker values between consecutive time point biological samples. While not wishing to be bound by any particular theory of operation, it has been found that re-testing an immediately previously isolated sample rather than merely relying on the information provided when that sample was originally tested reduces the inherent error in the analysis, thereby providing enhanced accuracy.

It is noted, therefore, that the presently disclosed methods are particularly applicable for measurements of parameters where the relevant differences are ones of degree. The presently disclosed methods are those less applicable (although not inapplicable) to parameters where presence or absence in a sample is the readout of interest.

As used herein, the term “biomarker” refers to any testable characteristic of a sample (e.g., a biological sample) for which an assay exists allowing for a parameter of that testable characteristic to be determined in the sample. Exemplary biomarkers include concentrations of the biomarker in the sample, which also includes for example an expression level of a gene product, either a nucleic acid gene product or a protein gene product. Biomarkers thus include without limitation gene expression levels, levels of modified gene products (e.g., post-translationally and/or otherwise modified proteins, particularly wherein the post-translational and/or other modifications are indicative of a disease or health state), levels of metabolites, etc.

In some embodiments, the biomarker is an expression level of a gene product, optionally, a protein, peptide, nucleic acid, or a fragment thereof or is an amino acid or nucleic acid sequence thereof. As would be understood by one of ordinary skill in the art, any assay can be used to determine a concentration or other characteristic of the biomarker in a sample. By way of example and not limitation, mass spectroscopy (MS), including but not limited to liquid chromatography mass spectrometry (LC-MS), can be employed to determine concentrations, and in some embodiments can be followed by sequencing in order to determine additional characteristics of a biomarker in a sample. LC-MS, for example, can be employed to determine the relative amounts of molecules ranging in size from tens of daltons (e.g, for small molecules) to hundreds of thousands of daltons (e.g., for biomolecules).Other isolation and/or purification techniques including but not limited to ELISA, affinity chromatography including but not limited to Immobilized Metal Ion Affinity Chromatography (IMAC), etc. Where a biomarker is a nucleic acid, quantification techniques including but not limited to quantitative polymerase chain reaction (qPCR) can be employed, including but not limited to quantitative reverse transcription polymerase chain reaction (qRT-PCR).

Once a characteristic (e.g., a concentration) of a biomarker is determined, a representation of a difference between the characteristic in a first sample and the same characteristic in a second sample can be determined. In some embodiments, the difference is expressed as a percent change from one to the next, although percent change is not the only way to express such a difference. In some embodiments, this difference (e.g., the percent change and/or other metric) can be outputted to a visible medium (e.g., printed, displayed on a screen either graphically or in raw form, etc.).

Given that samples will need to be retained so that they can be re-tested at lo a later date and compared to subsequently isolated samples, in some embodiments an isolated sample is stored under conditions expected to retain the integrity of characteristic of interest. For biological samples, this can include storing the sample at reduced temperature including but not limited to −20° C. or less, −70° C. or less, or around −200° C. (i.e., storage in liquid nitrogen). As desired, various preservatives can also be added to a biological sample for storage, provided that the presence of the preservative(s) does not affect the characteristic of interest once the sample is removed from storage for re-testing.

### EXAMPLE

The following Example provides illustrative embodiments. In light of the present disclosure and the general level of skill in the art, those of skill will appreciate that the following Example is intended to be exemplary only and that numerous changes, modifications, and alterations can be employed without departing from the scope of the presently disclosed subject matter.

### EXAMPLE

### Patient Breast Biomarker Concentration Measurements

MUC1 (Mucin1), a membrane tethered glycoprotein, is over-expressed and aberrantly glycosylated in over 90% of BC cases. Tumor-associated MUC1 (tMUC1) is a marker of an aggressive phenotype that is cleaved from epithelial cells and released into circulation, allowing detection in the serum. TABOO4 is a monoclonal antibody that specifically detects tMUC1 expression in human breast cancer tissue from all major subtypes and does not detect normal MUC1 that is present on most epithelial cells. The AGKURA® Personal Score (APS) Enzyme Immunoassay (EIA) is a sandwich solid phase enzyme-linked immunosorbent assay (ELISA) that specifically detects tMUC1 (Roy et al. (2017) A tumor specific antibody to aid breast cancer screening in women with dense breast tissue. Genes & Cancer 8(3-4):536-549). The assay uses two forms of the TABOO4 monoclonal antibody that specifically recognize an epitope on the peptide core of tumor-associated mucin 1 or tMUC1. TABOO4c (chimeric) antibody is used for solid phase immobilization (on the microtiter wells) while TABOO4m (mouse) antibody conjugated to horseradish peroxidase (HRP) is used to make the antibody-enzyme conjugate solution.

During this assay, the test sample was allowed to react first with the immobilized TABOO4c at 25° C. for 1 hour. The wells were washed with Wash Buffer to remove unbound antigen. The TABOO4mHRP conjugate was then added for 1 hour at 25° C. allowing it to react with the immobilized antigen, which resulted in tMUC1 being sandwiched between the solid phase and the enzyme-linked antibodies. The wells were washed to remove unbound labeled antibodies. TMB reagent, which detects horseradish peroxidase (HRP) activity, was added and incubated for 20 minutes, resulting in the formation of a blue color. This color development was stopped with the addition of Stop Solution changing the color to yellow.

The standard used by the APS ELISA was a protein lysate isolated from a MUC1-expressing tumor cell line (KCM), which was also used to generate the TABOO4 antibody. The concentration of tMUC1 was directly proportional to the color intensity of the test sample. Absorbance was measured spectrophotometrically at 450 nm and used to calculate tMUC1 concentration relative to the KCM standard, which was the AGKURA® Personal Score.

Table 1 compares tMUC1 concentrations measured in two patients at two time points, each using APS ELISA kits from two separate batches manufactured under GMP. Biomarker control materials (BIO-RAD Liquichek Tumor Marker Quality Controls) were within the defined acceptable QC range based on standard laboratory practice (Mean±2.58a) indicating both sets of measurements passed Quality Control rules and results were acceptable. All measurements correspond to triplicate wells.

TABLE 1

| | tMUC1 Concentrations (μg/ml) | | | |
|---|---|---|---|---|
| | Patient A | | Patient B | |
| | ELISA Plates* | | ELISA Plates* | |
| Timepoint (Sample) # | 1 | 2 | 1 | 2 |
| 1 | 39.6 | 53.1 | 31.4 | 32.7 |
| 2 | 61.6 | 81.3 | 27.7 | 28.8 |
| % Change using previous | 56% | 53% | −12% | −12% |
| sample as comparator on | | | | |
| same plate | | | | |

| | | |
|---|---|---|
| % Change across plates | 105% | −8% |
| (Timepoint 1 Plate 1; | | |
| Timepoint 2 Plate 2) | | |
| % Change across plates | 16% | −15% |
| (Timepoint 1 Plate 2; | | |
| Timepoint 2 Plate 1) | | |

### Discussion of the Example

Clinicians often use serial monitoring of biomarkers in patients under treatment to guide clinical decisions. Thus it is imperative that the relative change in the biomarker concentration (tMUC1 in this example illustration) is accurate. Table 1 illustrates results from two patients who were being treated for breast cancer. While the trends of the biomarker change were accurate for both patients, the relative magnitude of the change can be significantly different depending on the combination of plates used in this analysis. For example, for patient A, the two combinations of plates would indicate either a modest increase in the biomarker concentration of 16% or a remarkable increase of 105%. The results for patient B were more or less similar regardless of plate combinations used in the analysis. Interestingly, the increase in biomarker concentration was very consistent when the same plate was used to compare the two time points: 56% or 53% increase for patient A and a 12% decrease for patient B. Note that the plates and all reagents were manufactured using good manufacturing practices (GMP) and each concentration tabulated was on the basis of three (3) well measurements. Further, the tumor marker controls were within the control limits for all plates implying the results were acceptable per current laboratory practices. While results from two patients are presented in Table 1, the consistency of the relative change in biomarker concentration measured on the same plate between timepoints has been demonstrated in dozens of healthy volunteers and cancer patients.

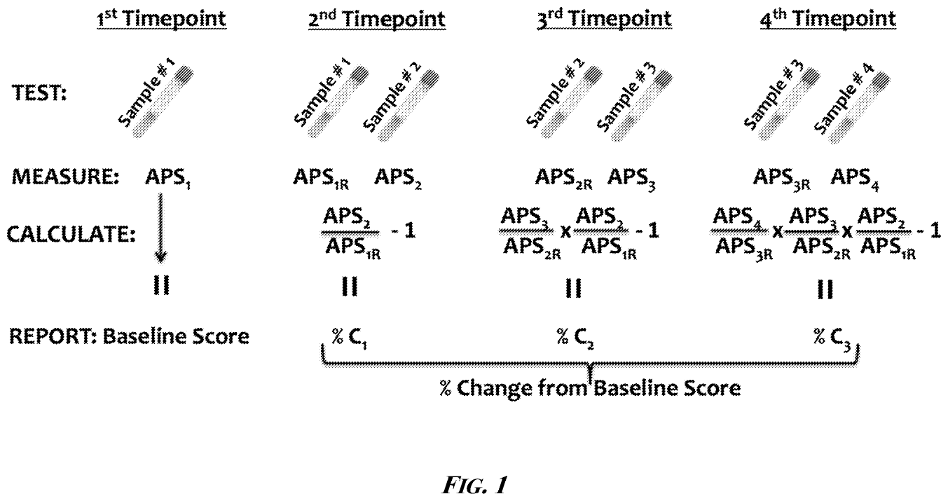

These observations led us to develop a comparator approach to serial biomarker measurements where the immediately prior timepoint sample was used as a comparator for the new timepoint sample (FIG. 1). The samples were stored at −80° C. between measurements, and we have demonstrated no deterioration of biomarker concentrations with multiple freeze-thaw cycles. Nonetheless, the process was designed to minimize the freeze-thaw cycles and each timepoint sample was only tested twice as illustrated in FIG. 1. The absolute concentration was only reported for the first timepoint. This is referred to as the Baseline Score. All subsequent measurements are reported as, for example, a percent change from the Baseline Score per the ratio calculation approach shown in FIG. 1. It is noted, however, that other expressions of differences between the “current” sample and its immediate predecessor can also be employed, including but not limited to fold-change, absolute measurement values, etc.

While this approach was developed for OncoTAb's TAB004-based ELISA (AGKURA® Personal Score or APS; OncoTAb, Inc., Charlotte, N.C., United States of America), the approach can also be used for any laboratory test for which a clinician desires accurate measurement of change in biomarker concentrations (proteins, DNA, and DNA fragments) from one sample to a subsequent sample. The subscripts 1, 2, 3 etc. refer to the timepoint and the ‘R’ extension to the subscript is used when the sample is used as a comparator with the subsequent timepoint sample. While FIG. 1 only illustrates four timepoints, there is no limit to the number of timepoints that can be employed using this approach. Further, even though the previous examples we have given are for cancer patients being monitored for response to treatment, this approach is also applicable for screening for cancer; monitoring during treatment; monitoring for recurrence as well as other diseases with a strong correlation with biomarker values/trends.

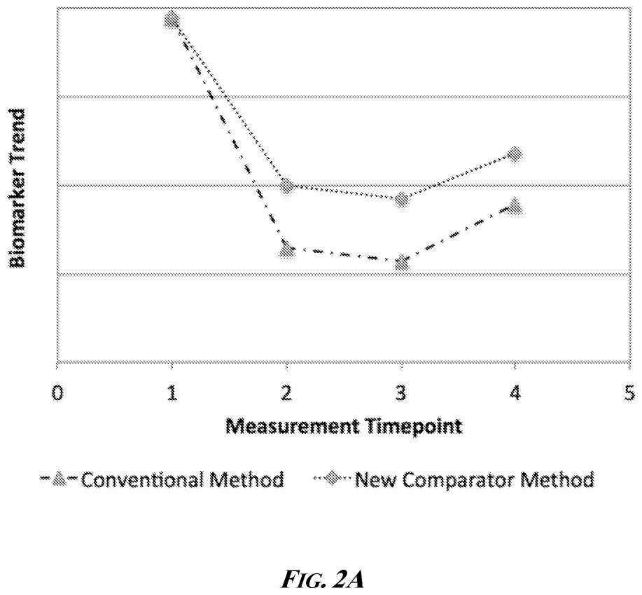

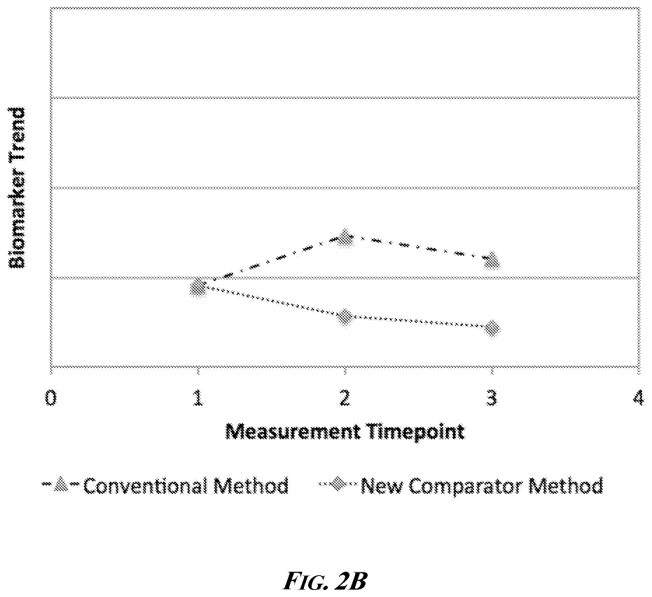

The above-described method was applied to the serial timepoint sample measurements from two additional patients: (a) a patient undergoing breast cancer treatment; and (b) a patient being monitored for recurrence of breast cancer. The biomarker trend line from the conventional method of reporting absolute measurements were compared to the trend line from the new process of using the previous timepoint sample from the patient as a comparator in FIG. 2. The conventional method reported the absolute value of measurements as measured. In order to construct the trend line for the comparator method, the % changes between consecutive measurements were used to calculate concentration values at each time point. For example, for the 2nd time point, the calculated concentration was: (% C1+1)×APS1 where % C1 is the % change between the 2nd time point and the baseline score as per formula shown in FIG. 1 and APS1 was the baseline score. Similarly, for the 3rd time point, the calculated concentration was: (% C2+1)×APS1 where % C2 is the % change between the 3rd time point and the baseline score as per formula shown in FIG. 1. This way, a concentration was calculated for each time point using the % change values between consecutive time points. In essence, the comparator method disclosed herein completely removed the analytical ELISA variation, and its trend line showed the true biological variation for that patient. The significance of this was borne out by the 2 examples in FIGS. 2A and 2B as discussed below.

While for patient (a) the overall trends appeared similar, for patient (b), the trends were dramatically different. The conventional method showed an increase from timepoint 1 to timepoint 2 in patient (b) while the new comparator methods showed a decrease. This difference in trendlines was due to the fact that the biological variation in this patient (b) from timepoint 1 to timepoint 2 was smaller than the analytical ELISA variation. For all the measurements shown in FIG. 2, the tumor marker control was within the mean ±2.58σ limits. For patient (a), the biological variation was much larger than the analytical ELISA variation given the significant drop between timepoints 1 and 2, and hence the overall trendlines looked similar between the two methods even though the magnitude of the changes was different. These results underscored the importance of using the immediate previous timepoint sample as a comparator when there was a need to accurately detect changes in biomarker trends (i.e. increasing or decreasing with time).

It will be understood that various details of the presently disclosed subject matter may be changed without departing from the scope of the presently disclosed subject matter. Furthermore, the foregoing description is for the purpose of illustration only, and not for the purpose of limitation.

1. A method for serial monitoring of a biomarker using a comparator approach, the method comprising:
(a) providing a first timepoint measurement with respect to a biomarker present in a first biological sample isolated from a subject;
(b) obtaining subsequent (2^{nd}, 3^{rd}, 4^{th} etc.) timepoint measurements for the same biomarker in subsequent (2^{nd}, 3^{rd}, 4^{th} etc.) biological sample isolated from the subject;
(c) retesting the immediately previous biological sample with respect to the biomarker along with the subsequent timepoint biological samples to obtain a comparative change in the biomarker between time points (1^{st} sample retested with 2^{nd} sample; 2^{nd} sample retested with 3^{rd} sample; 3^{rd} sample retested with 4^{th} sample; etc.);
(d) calculating a percent change and/or other metric in to biomarker between consecutive timepoint biological samples (1^{st} sample retested with 2^{nd} sample; 2^{nd} sample retested with 3^{rd} sample; 3^{rd} sample retested with 4^{th} sample; etc.); and
(e) calculating the biomarker concentration at each time point with respect to the first time point measurement using the percent change and/or other metric of biomarker values between consecutive time point biological samples.

2. The method of claim 1, further comprising outputting the percent change and/or other metric of biomarker values to a visible medium.

3. The method of claim 1, wherein the biomarker is an expression level of a gene product, optionally, a protein, peptide, nucleic acid, or a fragment thereof or is an amino acid or nucleic acid sequence thereof.

4. The method of claim 1, wherein the method employs an ELISA assay.

5. The method of claim 1, wherein the method employs a real-time or quantitative PCR to determine the relative amounts of a known sequence.

6. The method of claim 1, wherein the method employs liquid chromatography mass spectrometry (LC-MS) to determine the relative amounts of molecules ranging in size from tens of daltons (small molecules) to hundreds of thousands of daltons (biomolecules).
